(19) United States
(12) Patent Application Publication
(10) Pub. No.: US 2014/0207248 A1
(43) Pub. Date: Jul. 24, 2014

(54) HIERARCHICAL MULTISCALE FIBROUS SCAFFOLD VIA 3-D ELECTROSTATIC DEPOSITION PROTOTYPING AND CONVENTIONAL ELECTROSPINNING

(71) Applicant: THE TRUSTEES OF THE STEVENS INSTITUTE OF TECHNOLOGY, Hoboken, NJ (US)

(72) Inventors: Hongjun Wang, Millburn, NJ (US); Gerald Riccardello, Succasunna, NJ (US); Jiale Li, Cresskill, NJ (US)

(73) Assignee: THE TRUSTEES OF THE STEVENS INSTITUTE OF TECHNOLOGY, Hoboken, NJ (US)

(21) Appl. No.: 14/157,602

(22) Filed: Jan. 17, 2014

Related U.S. Application Data
(60) Provisional application No. 61/754,203, filed on Jan. 18, 2013.

(51) Int. Cl. A61F 2/02

(52) U.S. Cl. CPC A61F 2/02
USPC 623/23.72; 264/401

(57) ABSTRACT

A hierarchical multiscale fibrous scaffold comprises multiple patterned layers of microfibers with one or more layers of nanofibers interleaved therebetween. In a method for making such scaffolds, electrodeposition or near-field electrospinning is used to deposit patterned layers of microfibers in a stack. Conventional electrospinning is used to deposit nanofibers on the layers of microfibers. The method may be used to tune the mechanical properties of the scaffold, facilitated by microfibers, and the biological features of the scaffold, facilitated by nanofibers. Scaffolds produced by such a process may have highly biomimetic architectures, and allow rapid cellular infiltration and sustainable cell growth for multiple tissue types.

### CROSS-REFERENCE TO RELATED APPLICATIONS

The present application claims the benefit of U.S. Provisional Patent Application No. 61/754,203, filed on Jan. 18, 2013, the disclosure of which is incorporated by reference herein.

### FIELD OF THE INVENTION

The present invention relates to scaffolds for implantable tissue grafts, and, more particularly, to hierarchical multiscale fibrous scaffolds for tissue growth that are produced by 3-D electrostatic deposition prototyping and conventional electrospinning.

### BACKGROUND OF THE INVENTION

One of the current challenges in tissue repair/regeneration is to fabricate a scaffold that will promote cell infiltration with the necessary cues for cell differentiation and appropriate mechanical support for tissue formation. Evidence increasingly shows that nanofibers provide favorable environments for cell attachment and spreading, most likely as a result of their high surface area and their morphological and dimensional similarity to certain types of native extracellular matrix (ECM). However, conventional electrospinning can only create dense mats of nanofibers with small pore size (e.g., pore sizes less than about 5 μm) which discourage cell infiltration.

Thus, nanofiber mats are only used as two-dimensional (“2-D”) culture substrates or as meshes. However, it is preferable that tissue formation scaffolds provide a three-dimensional (“3-D”) environment for cell growth. Further, the opportunity to increase the pore size of nanofibers meshes or mats by manipulating fiber diameters is very limited. In this regard, attempts have been made to improve cell infiltration by using enzyme-degradable natural polymers, or co-electrospinning with sacrificial nanofibers, which are subsequently removed from the scaffold to generate large pores. Recent methods used in enlarging the pore size of electrospun nanofibers include salt leaching, the use of solid crystals on the nanofiber collection devices, wet electrospinning on a bath collector, combinations of nanofibers and microfibers, and laser/UV irradiation and electric fields for controlling deposition of nanofibers. The aforesaid methods, as they are presently applied, also weaken the mechanical strength of the nanofibers. Further, the native ECM fibers normally exist as a multiscale fibrous network composed of nanofibers and microscale bundles, in order to achieve both high mechanical strength provided by the bundles, and high surface area provided by the nanofibers for cell adhesion.

### SUMMARY OF THE INVENTION

An embodiment of the present invention provides a 3-D multiscale fibrous scaffold comprising patterned layers of microfibers and one or more layers of nanofibers between layers of microfibers. In embodiments of the scaffold, the patterns are ordered patterns of microfibers (e.g., parallel microfibers). In embodiments of the invention, adjacent layers of microfibers are arranged in an antiparallel fashion. The scaffold of the present invention overcomes the current limitations of pore size and cellular infiltration present in nanofibrous mats, while keeping the advantages presented by nanofibrous mats, such as high surface areas for cellular adhesion and the advantages of microfibrous layered structured, such as large pore size for cellular infiltration and structural support for tissues grown on the scaffolds.

In an embodiment of a method according to the present invention, a scaffold is fabricated as a stack of patterned layers of microfibers deposited by near-field electrospinning (also referred to herein as electrodeposition). In embodiments of the method, a layer of nanofibers is deposited on a patterned layer of microfibers by electrospinning, then another patterned layer of microfibers is deposited on the layer of nanofibers by near-field electrospinning. In embodiments of the method, the sequence of depositing a layer of nanofibers on a patterned layer of microfibers, followed by depositing another layer of microfibers on the layer of nanofibers is performed multiple times. In some embodiments, the patterning of microfibers and/or of nanofibers is performed by moving a collector plate beneath the needle tip of the electrospinning apparatus. In some embodiments, the collector plate is moved to change the distance between the needle tip and the collector plate before the layer of microfibers or the layer of nanofibers is deposited. In some embodiments, the movement of the collector plate is controlled by a computer program.

### BRIEF DESCRIPTION OF THE DRAWINGS

For a more complete understanding of the present invention, reference is made to the following detailed description of an exemplary embodiment considered in conjunction with the accompanying drawings, in which:

FIG. 1 is a conceptual illustration of an embodiment of an electrospinning apparatus for the fabrication of a multiscale hierarchical scaffold according to an embodiment of the present invention;

FIG. 2 is a schematic illustration of a step in a method of fabricating a multiscale hierarchical scaffold according to an embodiment of the present invention;

FIG. 3 is a schematic illustration of another step of the method of FIG. 1;

FIG. 4 is a schematic illustration of yet another step of the method of FIG. 1;

FIG. 5 is a schematic illustration of a further step of the method of FIG. 1;

FIG. 6 is a schematic illustration of yet a further step of the method of FIG. 1;

FIG. 7 is a microscopic image of a scaffold according to an embodiment of the present invention;

FIG. 8 is a second microscopic image of the scaffold of FIG. 7;

FIG. 9 is a microscopic image of a second scaffold according to an embodiment of the present invention;

FIG. 10 is a second microscopic image of the scaffold of FIG. 9;

FIG. 11 is a fluorescent microscopic image of the scaffold of FIG. 7 after seeding with cells and an incubation period of 1 day;

FIG. 12 is a fluorescent microscopic image of the scaffold of FIG. 8 after seeding with cells and an incubation period of 1 day;

FIG. 13 is a fluorescent microscopic image of the scaffold of FIG. 7 after seeding with cells and an incubation period of 7 days;

FIG. 14 is a fluorescent microscopic image of the scaffold of FIG. 8 after seeding with cells and an incubation period of 7 days;

FIG. 15 is a scanning electron microscopic (SEM) image of a third scaffold according to an embodiment of the present invention;

FIG. 16 is a SEM image of a fourth scaffold according to an embodiment of the present invention;

FIG. 17 is a SEM image of a fifth scaffold according to an embodiment of the present invention;

FIGS. 18A-18F are black-line drawings of microfibers according to embodiments of the present invention prepared by near-field electrospinning; and

FIG. 19 is a black-line drawing a sixth scaffold according an embodiment of the present invention prepared by near-field electrospinning.

### DETAILED DESCRIPTION OF THE EXEMPLARY EMBODIMENT

Embodiments of the present invention provide multiscale fibrous scaffolds with both microfibers and nanofibers, or with microfibers alone, and a method for making same. The microfibers provide mechanical strength and large pores for cell infiltration, and the nanofibers provide surfaces for cell adhesion. The microfibers are provided in size ranges that mimic fiber bundles of native extracellular material (“ECM”) (e.g., microfibers having diameters in the range of 1-30 μm. In a method of the present invention, three-dimensional (“3-D”) direct printing is used to precisely control the pore size between the microfibers (i.e., the interfiber distance). In some embodiments, nanofibers are integrated into the scaffold as distinct layers by electrospinning, whether the nanofibers are deposited randomly or in organized arrangements.

The use of near-field electrospinning (also referred to herein as “electrodeposition”) to fabricate polymer microfibers and conventional electrospinning to form nanofibers are known in the art. The process of electrospinning can be defined as the application of a high voltage source to a needle tip causing a solution of polymer, or other substance, to form a polymer jet that travels through an electrical field to deposit on a collector. In conventional applications, the polymer jet is unstable and spins randomly, forming a highly porous mesh of non-woven, sub-micron diameter polymer fibers with an inherently large surface area-volume ratio, large porosity, and small pore size. The fibers (also referred to as “nanofibers”) are randomly oriented within the mesh.

The process of near-field electrospinning uses a shorter distance between the needle tip and collector plate than conventional electrospinning, eliminating the spinning region of the liquid jet, making the process a direct, continuous-write technique for depositing highly-ordered polymer fibers.

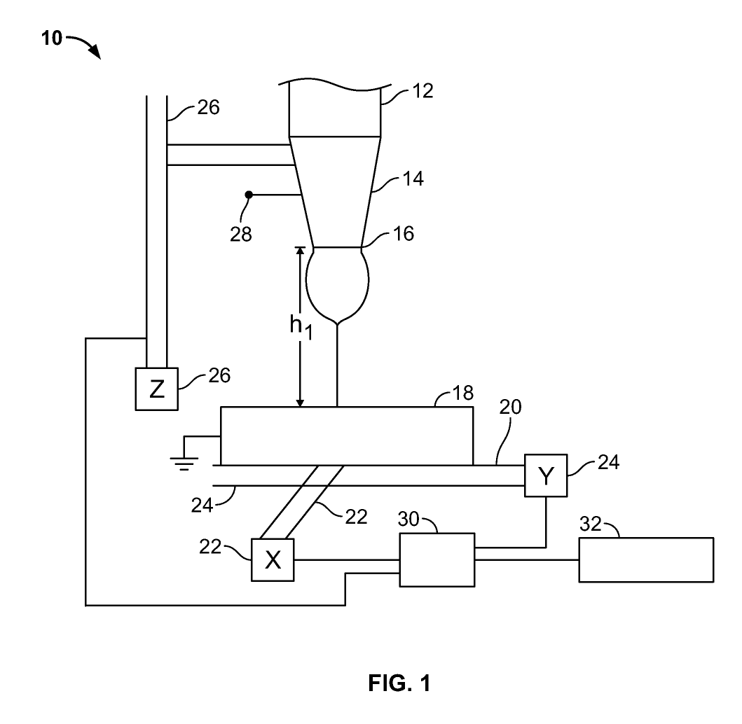

An embodiment of a method of making the scaffolds of the present invention involves the use of near-field electrospinning as a 3-D printing method to fabricate 3-D porous matrices with highly ordered microfibers (i.e., fibers having diameters in the range, for example, of 1-100 μm) in combination with layers of nanofibers (i.e., fibers having diameters in the range, for example, of 1-1000 nm). FIGS. 1-6 illustrate a process for 3-D printing/prototyping of a scaffold of microfibers via a near-field electrostatic deposition method according to an embodiment of the present invention. In contrast to the conventional uses of near-field electrospinning for controlling the collection of nanofibers on grounded surfaces, the method of the present invention involves the printing of microfibers over layers of previously deposited microfiber layers, with or without layers of nanofibers between layers of microfibers. The method thus builds microfibrous scaffolds with interconnected pores and customizable architectures.

### Hierarchical Multiscale Fibrous Scaffold

Hierarchical multiscale fibrous scaffolds are fabricated by 3-D printing of polymer microfiber layers into a porous 3-D scaffold, with a nanoscale fibrous component present between the layers. Such hierarchical multiscale scaffolds are a hybrid of microfibers, which are 3-D printed, and nanofibers which are produced by conventional electrospinning.

The microfibers and nanofibers of the present invention may be made from a variety of materials, including polymers and polymers with additives. Suitable materials include, without limitation, polycaprolactone (PCL), polylactic acid (PLA), copolylactic acid/glycolic acid (PLGA), and blends thereof, and blends of polymers with other substances, such as, without limitation, PCL/collagen, PCL/chitosan, PCL/tissue extract, or PCL/chitosan/hydroxyapatite. The polymers and blends discussed above, as well as others, may also contain bioactive substances, such as small molecule drugs (e.g., TRITC, FITC), proteins or peptides (e.g., BSA, VEGF), or biocompatible dyes, any of which may be released in vivo in a controlled fashion as the implanted scaffold degrades.

In principle, the microfibers and nanofibers used to fabricate the multiscale scaffolds of the present invention may be electrodeposited or electrospun from solutions having sufficient viscosity that the fibers remain substantially intact during the fabrication process. In an exemplary embodiment, the solution has a viscosity similar to that of 10% to 15% (w/v) PCL in hexafluoroisopropanol (HFIP). Other solvents may used instead of, or blended with, HFIP, such as chloroform or acetic acid.

Turning to the figures, FIG. 1 is a conceptual illustration of an embodiment of an electrodeposition/electrospinning apparatus 10 for the automated fabrication of a multiscale hierarchal scaffold according to an embodiment of the present invention. The apparatus 10 is similar in construction and operation to electrodeposition/electrospinning apparatuses known in the art. In the apparatus 10, a syringe 12 is fitted with a needle 14 having a hollow needle tip 16 which is positioned over an electrically-grounded collector plate 18. The collector plate 18 is supported on a stage 20 which may be moved in three orthogonal dimensions by an X-direction manipulator 22, a Y-direction manipulator 24, and Z-direction manipulator 26. The syringe 12 is secured to the Z-direction manipulator 26 in a perpendicular position relative to the collector plate 18, with the needle tip 16 at a fixed distance h₁ above the collector plate 18. The collector plate 18 is electrically grounded, and a programmable high-voltage source 28 is attached to the needle 14. All manipulators 22, 24, 26 may be controlled by a programmable motor controller 30, and the steps of the fabrication process controlled by computer 32. A polymer solution is loaded into a syringe, with a needle tip, in a programmable infusion pump (not shown). The needle is held perpendicular to the collector plate and is secured to a Z-direction manipulator.

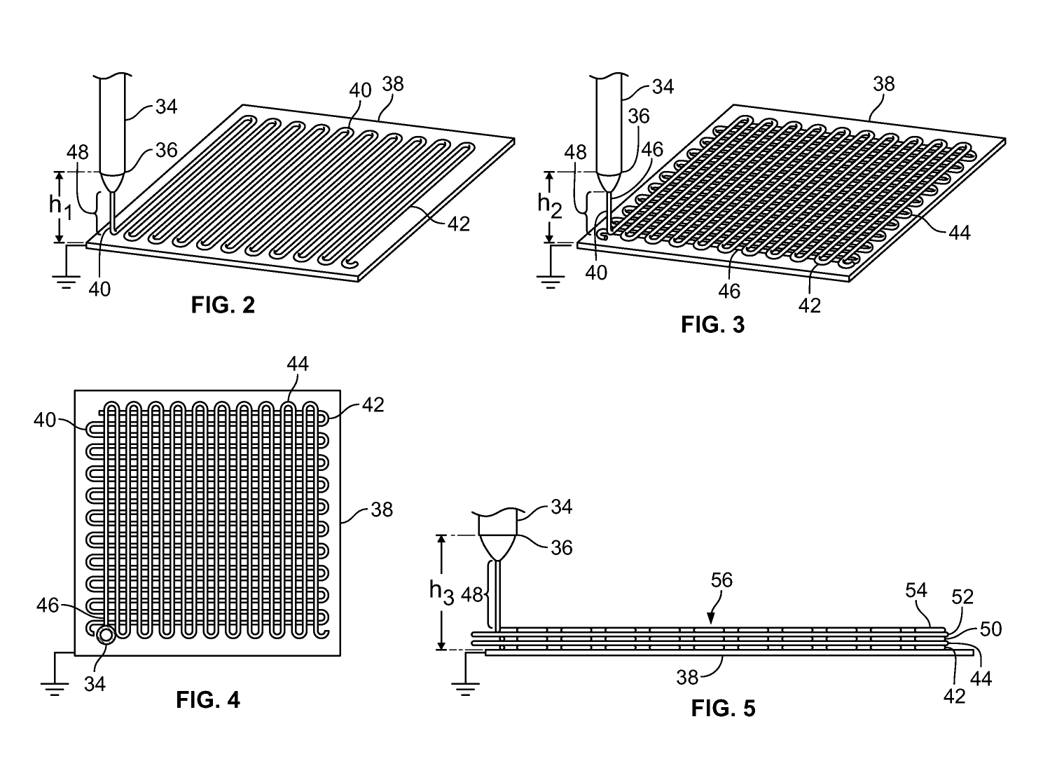

FIGS. 2-6 are schematic illustrations of steps in a method of preparing a multiscale fibrous scaffold according to an embodiment of the present invention. Referring first to FIG. 2, a syringe (not shown), fitted with a needle 34 having a needle tip 36, is placed into an electrodeposition/electrospinning apparatus (not shown) similar to apparatus 10 of FIG. 1. The needle tip 36 is positioned at a distance h₁ from the grounded collector plate 38. In some embodiments of the present invention, the distance h₁ is in the range of about 0.1 mm to about 5 mm. The syringe is loaded with the polymer solution used to form the microfibers 40. A voltage is applied to the needle tip 36, and the X-Y-Z manipulators (not shown) are activated to fabricate a patterned layer 42 of polymer microfibers 40. Voltages in the range of about 0.5 kV to about 5 kV are among those voltages suitable for producing microfibers by near-field electrospinning. Distances in the range of about 0.5 mm to about 3 mm are among those distances between the needle tip 36 and collectors plate 38 suitable for producing microfibers by near-field electrospinning.

Referring to FIG. 3, when the patterned layer 42 is complete, the collector plate 38 is lowered to increase the needle-to-collector plate distance to be increased to h₂, allowing space for a second patterned layer 44 of microfibers 46 to be formed. In the example of FIGS. 2-5, the distance h₂, voltage, and polymer flow rate are maintained at levels suitable for electrodeposition, which need not be the same levels used in depositing the patterned layer 42. Referring to FIGS. 3 and 4 together, the X-Y-Z manipulators are activated to fabricate the second patterned layer 44 of microfibers 46 on top of the patterned layer 42. In the example of FIGS. 2-5, the second patterned layer 44 is fabricated to be antiparallel to the first patterned layer 42.

While the polymer microfibers 46 are being deposited on the first patterned layer 42, airflow may be directed through a channel (not shown) which terminates in the electrodeposition zone 48. The air flow eliminates solvent from the electrodeposition zone, preserving the fibers underneath. In embodiments of the invention where a nanofibrous layer (not shown) is present, the use of air flow is particularly important for preserving the nanofibers of the nanofibrous layer when the microfibers are deposited thereupon.

Referring to FIG. 5, the deposition steps discussed with respect to FIGS. 2-4 may be repeated to fabricate layered scaffolds having a selected numbers of layers (see, e.g., layers 42, 44, 50, 52, 54 of the five-layered scaffold 56) by repetitively increasing the needle-to-collector distance h₃, while maintaining or adjusting the voltage and polymer flow rate.

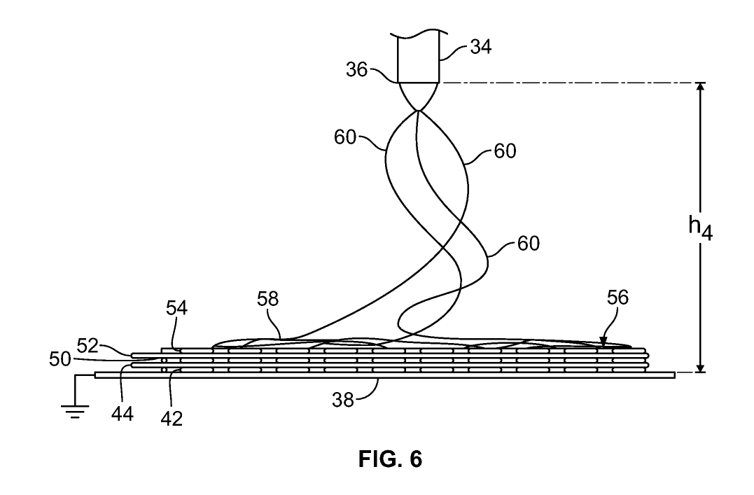

Referring to FIG. 6, in some embodiments of the present invention, a layer 58 of nanofibers 60 is deposited on a patterned layer of microfibers (e.g., patterned layer 54). The nanofibers 60 may be formed by electrospinning using the same apparatus 10 (see FIG. 1) used to form the patterned layers 42, 44, 50, 52, 54 of FIGS. 2-5, or a separate apparatus similar to apparatus 10 may be used. Additional patterned layers of microfibers may be deposited over the nanofiber layer 60. The scaffolds produced by the methods of the present invention may have layers of nanofibers between patterned layers of microfibers at multiple locations in the scaffold. More than one layer of nanofibers may be deposited over each other. Thus, a scaffold having stacks of patterned layers of microfibers interleaved with layers of nanofibers may be built in a repetitive process.

In an embodiment of the present invention, after a patterned layer of microfibers (e.g., patterned layer 54) is deposited, the collector plate 38 is lowered to increase the distance between the needle tip 36 and the collector plate 38 to a distance h₄, and the voltage and polymer flow rate are adjusted to values appropriate for electrospinning nanofibers 60. The nanofibers 58 are then spun and deposited onto the patterned layer 54. Voltages in the range of about 5 kV to 20 kV are among those voltages suitable for producing nanofibers by conventional electrospinning. Distances of about 8 cm to about 10 cm are among those distances between the needle tip 36 and collector plate 38 suitable for producing nanofibers by conventional electrospinning. In the example illustrated by FIG. 6, the nanofibers 60 are laid down in random orientations. By controlling the movement of the collector plate in the X-Y directions, the nanofibers can be laid down in an ordered pattern (e.g., aligned, cross-aligned, antiparallel, etc.).

In another embodiment of the method of the present invention, a layer of aligned nanofibers is prepared by electrospinning nanofibers onto the cylindrical surface of a rotating electrically-grounded circular disk, then cutting a portion of the collected nanofiber mat and placing it on a patterned layer of nanofibers. In the following exemplary embodiment, a circular iron disk having an axle therethrough is used in place of the collector plate and X-Y-Z manipulators discussed above with respect to FIG. 1. The disk may have a diameter in the range of about 76 mm to about 126 mm, and a thickness between its opposed faces in the range of about 15 mm to about 25 mm. The disk is arranged with its axis of rotation (i.e., through the axle) in a horizontal orientation, with the cylindrical surface of the disk in the range of about 100 mm to about 120 mm below the needle tip, and electrically grounded. The disk is then spun at a rate in the range of about 75 cycles/min to about 80 cycles/min, and a voltage in the range of about 10 kV to about 15 kV is applied to the needle. A PCL solution (about 8% of 80,000 MW PCL in HFIP) is delivered at a rate of about 10 μL/min, thereby depositing a layer of aligned electrospun nanofibers of PCL on the circular surface of the disk. After a sufficient amount of nanofiber has been collected (e.g., after a time of about 1 min to about 15 min), the layer of aligned nanofibers is cut free from the disk and applied to the upper surface of a patterned layer of microfibers, such as a patterned layer prepared according to the method illustrated in FIGS. 2 through 5 above. Once the layer of aligned nanofibers has been placed on the patterned layer of microfibers, a second patterned layer of microfibers may be deposited on the layer of aligned nanofibers, using, for example, the method of FIGS. 2-5.

The method of the present invention, as exemplified in FIGS. 1-5 may be used to fabricate highly-ordered 3-D fibrous scaffolds on the microscale (e.g., with microfibers having diameters of 3-30 μm) from a versatile range of polymers and polymers blended with other substances. The use of the near-field electrospinning allows the pore size, mechanical strength, and thickness of the scaffolds to be precisely controlled. The scaffolds may be customized with defined architectures for use with various bodily tissues. The method of the present invention may be used to fabricate 3-D multiscale fibrous scaffolds comprising microfibers and random and/or aligned nanofibers, such that the scaffolds have high porosity and interconnected pores to facilitate cell infiltration, and large surface areas for cell attachment. The selection of materials used to form the scaffolds allows control over the rate at which the scaffolds are degraded in vivo, and allows for the inclusion and controlled release of various large or small molecules (e.g., drugs, proteins, growth factors, minerals, buffers, or dyes). The method of the present invention can be used to fabricate scaffolds with unique properties (e.g., well-controlled pore size, high interconnectivity, tunable mechanical properties, and extracellular matrix-like topography, etc.). Such scaffolds, which are also embodiments of the present invention, allow for the regeneration and repair of both soft tissue and hard tissue. In embodiments of the present invention, repair of soft tissue includes repair of skin, muscles, tendons, ligaments, cartilage, hernia, blood vessels, nerves, etc. In embodiments of the present invention, repair of hard tissue includes repair of bone, cartilage, etc.

### Experimental Examples

The following discussions present non-limiting examples of certain embodiments of the methods and scaffolds of the present invention. Persons having ordinary skill in the relevant arts and possession of the present disclosure may make numerous modifications and variations on these embodiments without departing from the spirit and scope of the invention.

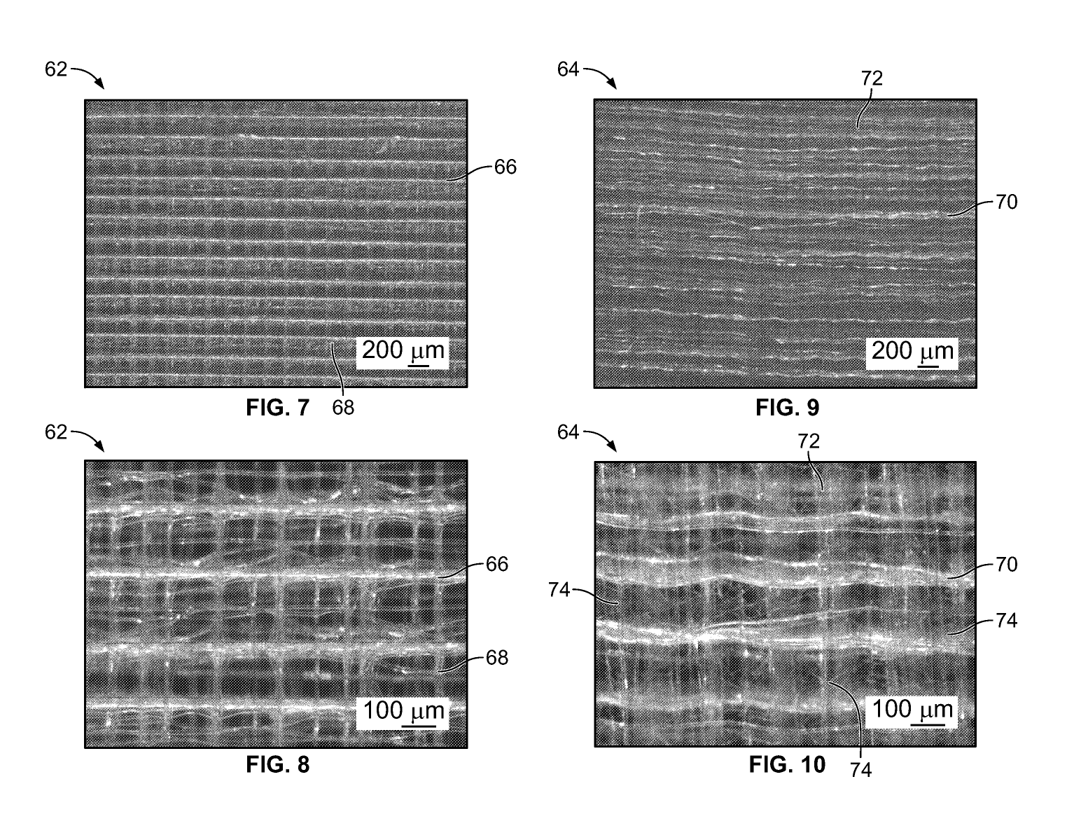

FIGS. 7-10 are microscopic images of scaffolds 62, 64 according to embodiments of the present invention. The scaffold 62 of FIGS. 7 and 8 consists of 44 layers of microfibers deposited in alternating antiparallel patterns (see, e.g, fiber 66 from a horizontal layer (relative to the orientation of the figure), and fiber 68 from an adjacent vertical layer). The interfiber spacing of the microfibers is about 200 μm, and the microfiber diameters are generally in the range of 10 μm to 20 μm. The scaffold 62 has a thickness of about 160 μm, and the microfiber diameters are generally in the range of 10 μm to 20 μm. The scaffold 64 of FIGS. 9 and 10 is a multiscale scaffold consisting of 44 layers of microfibers deposited in alternating antiparallel patterns (see, e.g., fiber 70 from a horizontal layer (relative to the orientation of the figure) and fiber 72 from an adjacent vertical layer), and layers of nanofibers (see, e.g., nanofibers 74 in FIG. 10), which were deposited after every two layers of microfibers. The interfiber spacing of the microfibers is about 200 μm, and the microfiber diameters are generally in the range of 10 μm to 20 μm. The scaffold 64 has a thickness of about 160 μm.

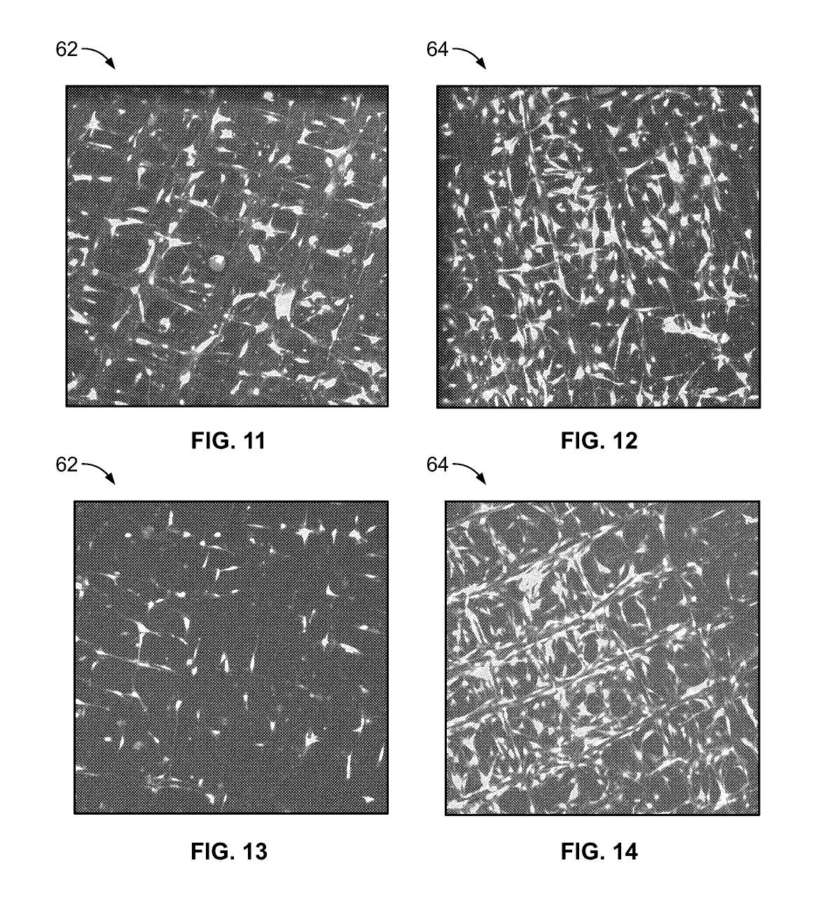

FIGS. 11-14 present fluorescent images of cells seeded onto the scaffolds 62, 64 of FIGS. 7-10 at day 1 and day 7 of incubation. The scaffolds 62, 64 were coated with collagen by immersion in a solution of 15 μg/mL of collagen in ethyl alcohol, followed by drying at room temperature, to improve cell adhesion. Each collagen-coated scaffold 62, 64 was seeded with about 43,000 green fluorescent protein (GFP)-transfected mouse stromal cells (OP-9 cells). Cell growth on the scaffolds 62, 64 were visualized using confocal and fluorescent microscopy at Day 1 and Day 7 of incubation.

First and second 88-layer scaffolds (not shown) were prepared for cell seeding according to methods similar to those used to fabricate and seed scaffold 62 and scaffold 64, respectively (i.e., the first 88-layer scaffold consisted of antiparallel layers of microfibers, and the second 88-layer scaffold consisted of antiparallel layers of microfibers with a layer of nanofibers deposited after every two layers of microfibers). The interfiber spacing of the microfibers was about 200 μm, and the microfiber diameters were generally in the range of 10 μm to 20 μm. The first and second 88-layer scaffolds had thicknesses of about 350 μm. Each of the first and second 88-layer scaffolds was seeded with about 87,000 OP-9 cells. At Days 1 and 7 of incubation the 88-layer scaffolds were cross-sections and cell growth across the thickness of each 88-layer scaffold was visualized with H&E staining.

The results for of the confocal visualization of the scaffolds 62, 64 can be seen in FIGS. 11-14, wherein FIG. 11 shows the results for scaffold 62 at Day 1, FIG. 12 shows the results for multiscale scaffold 64 at Day 1, FIG. 13 shows the results for scaffold 62 at Day 7, and FIG. 14 shows the results for multiscale scaffold 64 at Day 7.

It can be seen that cells (visible as white or light gray spots in the FIGS. 11-14) attached to each scaffold 62, 64 as early as Day 1, with a greater number of cells attached to the multiscale scaffold 64, possible due to the greater surface area provided by the nanofibers. It was also observed that cells are able to completely infiltrate the first and second 88-layer scaffolds (not shown) as early as Day 1.

Comparison of FIGS. 12 and 14 shows that cells continued to proliferate on the multiscale scaffold 64 through at least Day 7. Similar proliferation of cells on scaffold 62 is not readily apparent through comparison FIGS. 11 and 13. It was also observed that live cells were present in the interiors of the first and second 88-layer scaffolds (not shown) at Day 7.

### Further Embodiments of the Invention

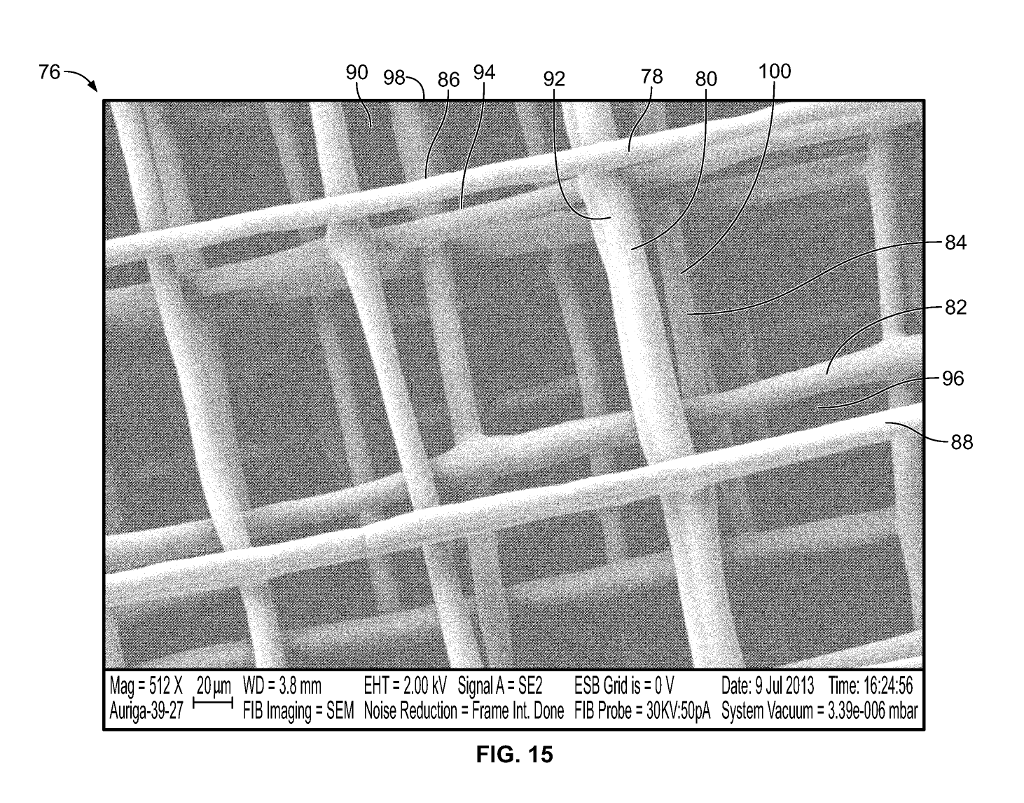

FIG. 15 is a scanning electron microscopic (SEM) image of a scaffold 76 according to an embodiment of the present invention. The scaffold 76 was prepared by an electrodeposition process according to an embodiment of the present invention. The scaffold 76 consists of a stack of multiple patterned microfiber layers (e.g., layers 78, 80, 82, 84) arranged in an antiparallel fashion. As can be seen in the SEM image, the microfibers (e.g., microfibers 86, 88, 90, 92, 94, 96, 98, 100) are precisely placed in antiparallel arrangements to define large (i.e., about 200 μm) spaces between the microfibers, providing the scaffold 76 with a high degree of porosity.

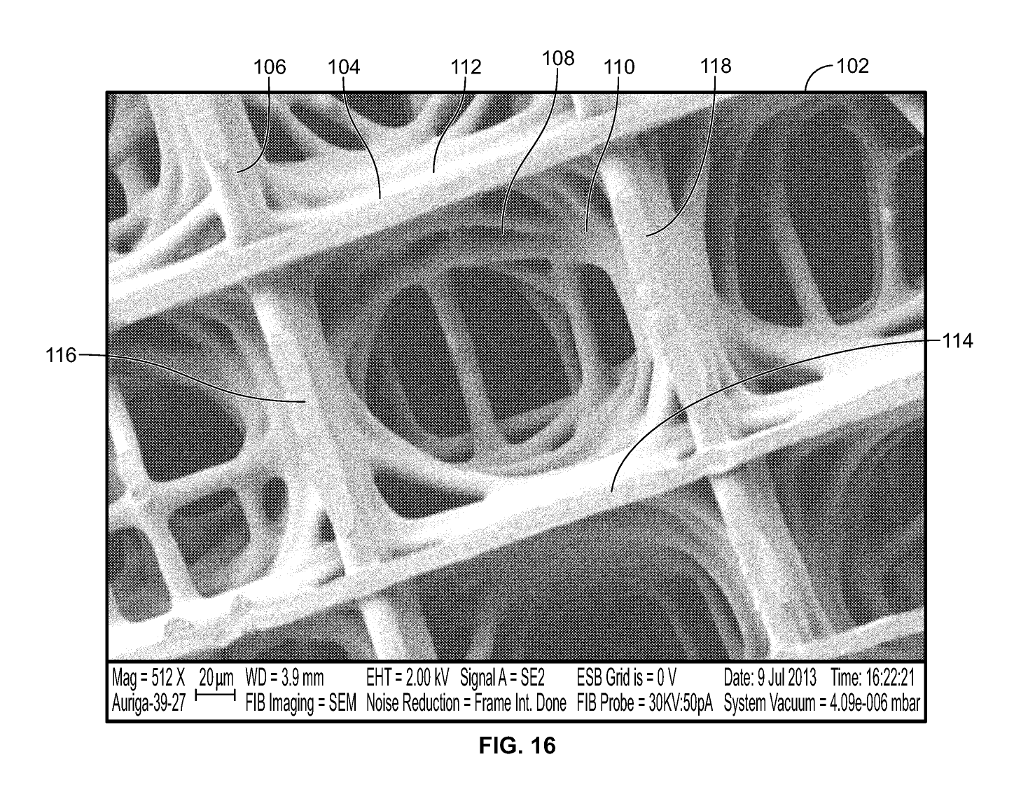

FIG. 16 is a SEM image of another scaffold 102 according to an embodiment of the present invention. The scaffold 102 was also prepared by an electrodeposition process according to an embodiment of the present invention. Two antiparallel microfiber layers 104, 106 are adjacent each other, and a coiled microfiber (e.g., coiled microfiber 108) is precisely placed on an opening (e.g., opening 110) defined by the microfibers 112, 114, 116, 118 of the layers 104, 106. The coiled microfiber 108 was shaped by controlled movement of the collector plate (not shown) during deposition of the microfiber 108.

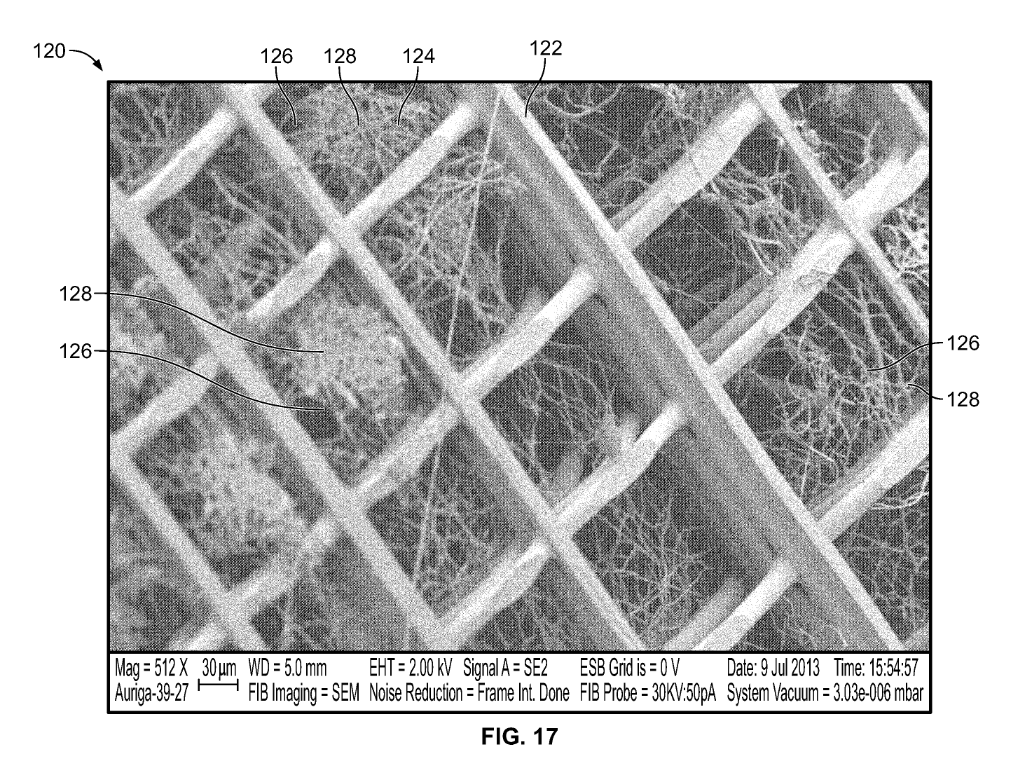

FIG. 17 is a SEM image of yet another scaffold 120 according to an embodiment of the present invention. Two antiparallel microfiber layers 122, 124 are adjacent each other, each having been prepared by an electrodeposition process according to a method of the present invention. A layer 126 of randomly-oriented nanofibers 128 has been deposited on the microfiber layer 124.

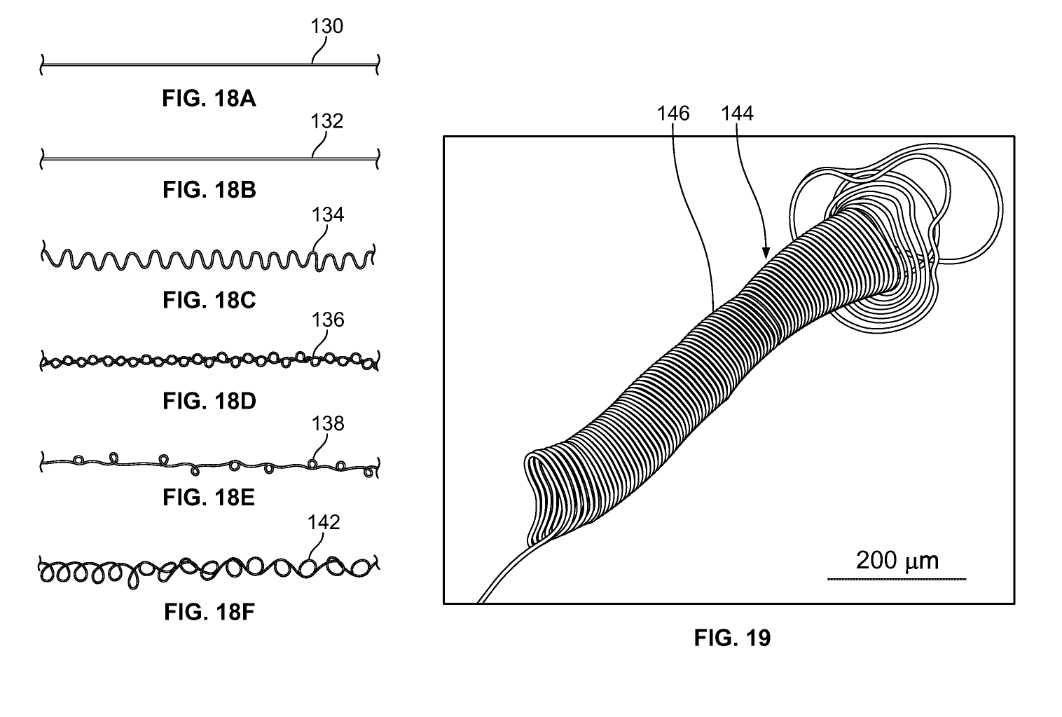

FIGS. 18A-18F are black-line representations of microfibers 130, 132, 134, 136, 138, 140 formed according to embodiments of the electrodeposition method of the present invention. These examples provide exemplary ranges of parameters that may be used in the fabrication of microfiber layers in scaffolds of the present invention.

Turning to FIG. 18A, the simple rectilinear microfiber 130 was formed using PCL in HFIP at a concentration in the range of about 6% to about 12% and a solution flow rate in the range of about 0.02 μL/min to about 0.06 μL/min. The distance between the needle tip and the collector plate was in the range of about 1.0 mm to about 2.0 mm, and the voltage applied across the needle tip and collector plate was in the range of about 1.3 kV to about 1.5 kV. The movement speed of the collector plate in the X direction was in the range of about 0.004 m/s to about 0.006 m/s. An air flow rate in the range of about 600 mL/min to about 1200 mL/min was used to remove solvent from the microfiber 130.

Turning to FIG. 18B, the bold simple rectilinear microfiber 132 was formed using PCL in HFIP at a concentration in the range of about 10% to about 14% and a solution flow rate in the range of about 0.02 μL/min to about 0.06 μL/min. The distance between the needle tip and the collector plate was in the range of about 2.0 mm to about 5.0 mm, and the voltage applied across the needle tip and collector plate was in the range of about 1.4 kV to about 2.0 kV. The movement speed of the collector plate in the X direction was in the range of about 0.001 m/s to about 0.002 m/s. An air flow rate in the range of about 500 mL/min to about 600 μL/min was used to remove solvent from the microfiber 132. The microfiber 132 has a larger diameter than the microfiber 130.

Turning to FIG. 18C, the wave-shaped microfiber 134 was formed using PCL in HFIP at a concentration in the range of about 10% to about 12% and a solution flow rate in the range of about 0.02 μL/min to about 0.04 μL/min. The distance between the needle tip and the collector plate was in the range of about 2.0 mm to about 4.0 mm, and the voltage applied across the needle tip and collector plate was in the range of about 1.5 kV to about 2.4 kV. The movement speed of the collector plate in the X-Y directions was in the range of about 0.002 m/s to about 0.004 m/s. An air flow rate in the range of about 300 mL/min to about 600 μL/min was used to remove solvent from the microfiber 132.

Turning to FIG. 18D, the closely-looped microfiber 136 was formed using PCL in HFIP at a concentration in the range of about 8% to about 12% and a solution flow rate in the range of about 0.02 μL/min to about 0.04 μL/min. The distance between the needle tip and the collector plate was in the range of about 2.0 mm to about 5.0 mm, and the voltage applied across the needle tip and collector plate was in the range of about 1.8 kV to about 2.6 kV. The movement speed of the collector plate in the X-Y directions was in the range of about 0.002 m/s to about 0.003 m/s. An air flow rate in the range of about 200 mL/min to about 400 mL/min was used to remove solvent from the microfiber 136.

Turning to FIG. 18E, the incompactly-looped microfiber 138 was formed using PCL in HFIP at a concentration in the range of about 8% to about 10% and a solution flow rate in the range of about 0.03 μL/min to about 0.05 μL/min. The distance between the needle tip and the collector plate was in the range of about 1.5 mm to about 3.0 mm, and the voltage applied across the needle tip and collector plate was in the range of about 1.6 kV to about 2.4 kV. The movement speed of the collector plate in the X-Y directions was in the range of about 0.004 m/s to about 0.006 m/s. An air flow rate in the range of about 200 mL/min to about 400 μL/min was used to remove solvent from the microfiber 138.

Turning to FIG. 18F, the large-looped microfiber 140 was formed using PCL in HFIP at a concentration in the range of about 8% to about 12% and a solution flow rate in the range of about 0.04 μL/min to about 0.08 μL/min. The distance between the needle tip and the collector plate was in the range of about 5.0 mm to about 10.0 mm, and the voltage applied across the needle tip and collector plate was in the range of about 2.4 kV to about 3.6 kV. The movement speed of the collector plate in the X-Y directions was in the range of about 0.002 m/s to about 0.004 m/s. An air flow rate in the range of about 200 mL/min to about 600 mL/min was used to remove solvent from the microfiber 140.

FIG. 19 is a black-line drawing of spiral tubular scaffold 142 of the present invention formed from a microfiber 144 according to a method of the present invention. The tubular scaffold 142 has an outer diameter of roughly 100 μm. It was formed using PCL in HFIP at a concentration in the range of about 10% to about 12% and a solution flow rate in the range of about 0.05 μL/min to about 0.10 μL/min. The distance between the needle tip and the collector plate was in the range of about 4.0 mm to about 10.0 mm, and was increased during the fabrication of the tubular scaffold 142. The voltage applied across the needle tip and collector plate was in the range of about 2.0 kV to about 3.6 kV. The collector plate was not moved in the X-Y directions, and no air flow was applied to the microfiber 144.

It will be understood that the embodiments of the invention described herein are merely exemplary and that a person skilled in the art may make many variations and modifications without departing from the spirit and scope of the invention. For instance, all such variations and modifications, in addition to those described above, are intended to be included within the scope of the invention, as embodied in the claims appended hereto.

1. A scaffold, comprising:
a plurality of patterned microfibrous layers of microfibers having diameters in the range of about 3 μm to about 30 μm; and
at least one nanofibrous layer including nanofibers having diameters in the range of about 1 nm to about 1000 nm, said nanofibrous layer being between and adjacent to two of said plurality of microfibrous patterned layers, wherein said plurality of microfibrous patterned layers and said at least one nanofibrous layer are arranged one on top of another in a stacked arrangement.

2. A method for fabricating a scaffold comprising electrospun fibers using an electrospinning apparatus having a hollow needle tip, a voltage means for applying a controllable voltage to the needle tip, an electrically-grounded collector plate, and a collector plate manipulating means for controllably moving the collector plate in the orthogonal x, y, and z directions, the voltage means and the collector plate manipulating means being controllable by a computer program, said method including the steps of:
depositing a first at least one microfiber having a diameter in the range of about 3 μm to about 30 μm onto the collector plate while electrospinning the first at least one microfiber from a polymer solution with a first voltage in the range of about 0.5 kV to about 5 kV applied to the needle tip by the voltage means and a first distance of about 0.5 mm to about 3 mm between the needle tip and the collector plate, and while moving the collector plate with the collector plate manipulating means such that the first at least one microfiber is deposited on the collector plate in an ordered pattern so as to form a first patterned microfibrous layer on the collector plate;
moving the collector plate with the collector plate manipulating means such that the distance between the needle tip and the collector plate is increased;
depositing a second at least one microfiber having a diameter in the range of about 3 μm to about 30 μm onto the first patterned microfibrous layer while electrospinning the second at least one microfiber from the polymer solution with a second voltage in the range of about 0.5 kV to about 5 kV applied to the needle tip by the voltage means and a second distance of about 0.5 mm to about 3 mm between the needle tip and the collector plate, and while moving the collector plate with the collector plate manipulating means such that the second at least one microfiber is deposited on the first patterned microfibrous layer in an ordered pattern so as to form a second patterned microfibrous layer on the first patterned microfibrous layer;
moving the collector plate with the collector plate manipulating means such that the distance between the needle tip and the collector plate is increased;
depositing at least one nanofiber having a diameter in the range of about 1 nm to about 1000 nm onto the second patterned microfibrous layer while electrospinning the at least one nanofiber from the polymer solution with a third voltage in the range of about 5 kV to about 20 kV applied to the needle tip by the voltage means and a third distance of about 8 cm to about 10 cm between the needle tip and the collector plate, such that the at least one nanofiber is deposited on the second patterned microfibrous layer so as to form a nanofibrous layer on the second patterned microfibrous layer;
moving the collector plate with the collector plate manipulating means such that the distance between the needle tip and the collector plate is decreased;
depositing a third at least one microfiber having a diameter in the range of about 3 μm to about 30 μm onto the nanofibrous layer while electrospinning the third at least one microfiber from the polymer solution with a fourth voltage in the range of about 0.5 kV to about 5 kV applied to the needle tip by the voltage means and a fourth distance of about 0.5 mm to about 3 mm between the needle tip and the collector plate, and while moving the collector plate with the collector plate manipulating means such that the third at least one microfiber is deposited on the nanofibrous layer in an ordered pattern so as to form a third patterned microfibrous layer on the nanofibrous layer, wherein the first, second, third and fourth voltages and the movement of the collector plate manipulating means in the aforesaid depositing and moving steps are controlled by the computer program.
